(19) United States
(12) Patent Application Publication
(10) Pub. No.: US 2015/0297458 A1
(43) Pub. Date: Oct. 22, 2015

(54) RAPID RECONSTITUTION PACKAGES FOR MIXING AND DELIVERY OF DRUGS

(71) Applicant: Noel M. Elman, Cambridge, MA (US)

(72) Inventor: Noel M. Elman, Cambridge, MA (US)

(73) Assignee: Massachusetts Institute of Technology, Cambridge, MA (US)

(21) Appl. No.: 14/696,788

(22) Filed: Apr. 27, 2015

Related U.S. Application Data
(63) Continuation of application No. PCT/US2013/066758, filed on Oct. 25, 2013.
(60) Provisional application No. 61/719,012, filed on Oct. 26, 2012.

(51) Int. Cl. A61J 1/20; A61M 5/315; F16K 37/00; A61M 5/28

(52) U.S. Cl. CPC A61J 1/2093; A61M 5/283; A61M 5/284; A61M 5/31596; F16K 37/0075; A61M 2202/0007; A61M 2205/02; A61M 2005/31598

(57) ABSTRACT

Rapid reconstitution package. The package includes a substantially cylindrical structure including a diluent chamber and a drug chamber communicating through a valve. The cylindrical structure is configured to retract under an axial force causing the valve to open to allow the diluent to flow through the valve into the drug chamber for mixing and reconstitution of the drug. The drug chamber shape is tuned to enhance mixing. In another aspect, the invention is a method for designing a rapid reconstitution package using computational fluid dynamics techniques.

### PRIORITY INFORMATION

This application claims priority to provisional application Ser. No. 61/719,012 filed on Oct. 26, 2012, the contents of which are incorporated herein by reference.

### SPONSORSHIP INFORMATION

This invention was made with government support under Contract No. W911NF-13-D-0001 awarded by the Army Research Office. The government has certain rights in the invention.

### BACKGROUND OF THE INVENTION

This invention relates to rapid reconstitution packages designed using computational fluid dynamics techniques to optimize the fluidic structure for mixing and delivery of lyophilized drugs.

Emergency medications in ambulatory settings are formulated and administered often in time-critical situations, leading to an increase in the probability of errors for therapeutic treatment [1-4]. Reconstituting lyophilized drugs is an operational challenge in demanding environments, e.g. disaster zones, battlefield support, and rural areas. Heat insulation is critical for biological drugs, e.g. antibodies, for which the efficacy of the active pharmaceutical ingredient (API) heavily relies on biomolecular conformality. Small molecule drugs consisting of pure chemical formulation are often deployed in liquid form in hermetically packaged glass ampules. In order to avoid hydrolysis, biologics are stored in lyophilized form in hermetically sealed glass vials that require subsequent reconstitution upon delivery. Lyophilization process involves freeze-drying the active pharmaceutical ingredient (API) combined with excipients, rendering a powder form with specific physical properties that include mass density, solubility, crystal size and packing factor.

The steps for drug reconstitution in vials require: injecting specific diluents into the vials, manual shaking and visually confirming homogeneity [5]. Various pre-filled dual-chamber solutions exist for on-demand reconstitution. These solutions typically rely on chemical solubility between a diluent and lyophilized drug and still require manual shaking. The design of these devices typically consists of two chambers: one for the drug in lyophilized form (solute) and another for the diluent (solvent). Dual-chamber devices have been utilized for small API payloads with a limited number of pharmacological therapies, such as human growth hormone (HGH) commercially known as Genotropin (Pfizer, Inc.) developed by Vetter. See, DE 3736343A1, CA 2397829C, and DE 102004056617A1 [6].

### SUMMARY OF THE INVENTION

In a first aspect, the invention is a rapid reconstitution package including a substantially cylindrical structure including a diluent chamber and a drug chamber communicating through a valve, the cylindrical structure configured to retract under axial force. The retraction causes the valve to open to allow the diluent to flow through the valve into the drug chamber for mixing and reconstitution of the drug, wherein the drug chamber shape is tuned to enhance mixing. An embodiment of this aspect of the invention includes a plunger operatively connected to the valve to open the valve under the axial force. It may be preferred that the cylindrical structure be sized to fit within a standard syringe body for activation. A separate mixing chamber may be provided to communicate with the drug chamber to provide for enhanced mixing. In another embodiment of this aspect of the invention, the drug chamber shape deflects a jet of diluent downwards for enhanced mixing. The jetting effect can be tuned both in intensity and spatial direction depending on the drug that is required to be reconstituted. In another embodiment the RRP disclosed herein can be used not only as a cartridge but as an injector or even an auto-injector when it is coupled to a pre-loaded spring. See, US 2013/0178823, US 2012/0101475, U.S. Pat. No. 4,394,863 and WO 1991016094.

A second aspect of the invention is a method for designing a rapid reconstitution package including prototyping and testing physical properties of a package structure. Computer computational fluid dynamics is used to model and numerically process results of the testing. Experimental data is used to modify fluidic structures to achieve enhanced mixing. In a preferred embodiment, the computational fluid dynamics provides quantitative techniques to optimize mixing parameters for drug mixing, advection and diffusion phenomena. In yet another preferred embodiment, the modified fluidic structure increases inlet velocity into the drug chamber in the package. The CFD analysis allows a change in the drug chamber silhouette so that it acts as both a storage and mixing chamber while indices of vorticity and concentration of the output as a function of time are monitored. Furthermore, depending on a targeted release profile it is possible to adjust the silhouette combined with activation time to adjust for an overall release profile per pharmacological therapy.

### BRIEF DESCRIPTION OF THE DRAWING

FIG. 1a is a cross-sectional, isomeric view of an embodiment of a rapid reconstitution package disclosed herein shown within a standard syringe.

FIGS. 1b, c, and d are cross-sectional views of an embodiment of the rapid reconstitution package disclosed herein.

FIG. 2 is a schematic block diagram illustrating the iterative design process of a rapid reconstitution package according an embodiment of the invention forming a constant feedback system.

FIG. 3 is an illustration of a simulation mesh used to discretize the rapid reconstitution package at very fine elements (one million voxels) for regional calculation.

FIG. 4 shows cross-sectional views of a portion of a rapid reconstitution package showing various locations for the position of drugs within a drug chamber.

FIG. 5 is a perspective view of an output collection system used to characterize experimentally the flow output of an embodiment of the rapid reconstitution package disclosed herein.

FIGS. 6a, b, and c show velocity magnitude contours showing the velocity of jet stream flow through several embodiments of the rapid reconstitution package disclosed herein.

FIG. 7a shows vorticity simulations for an embodiment of the rapid reconstitution package disclosed herein.

FIG. 7b is a graph of average vorticity versus time over a two second inject for different embodiments of rapid reconstitution packages as disclosed herein.

FIG. 8 are velocity magnitude contours from simulation results of drug mass at a final output through successive generations of rapid reconstitution packages.

FIG. 9a shows simulation results from numerical models.

FIG. 9b is a graph of concentration versus time showing simulated outflow concentration profiles at three different positions over four activation times.

FIG. 9c is a graph of drug exhausted versus activation time showing the percentage of tPA released over simulated activation times.

FIG. 10 is a graph of drug exhausted against tested payloads showing the percentage of payload delivered for different payload masses.

FIG. 11 is a bar graph showing concentration against storage condition showing ELISA stability characterization.

FIG. 12 is a graph of drug concentration against time comparing experimental and numerical results.

FIG. 13 is a bar graph showing concentration against storage condition for HPLC concentrations of tPA in tested samples.

### DESCRIPTION OF THE PREFERRED EMBODIMENT

In order to effectively reconstitute lyophilized or powder-based drugs with large payloads (e.g. antibiotics and biologic drugs in the order of tens of milligrams) and low solubility APIs, numerical models have been considered taking into account the physical properties of drugs and diluents. The challenge lies in the design of devices to perform optimal reconstitution incorporating parameterization models of APIs and diluents, and engineering fluidic structures in order to optimize mixing homogeneity and response time as a function of input shear flows [7]. The next generation of reconstitution devices for large payloads (10 mg-250 mg) and low solubility APIs will therefore require a more comprehensive design consolidation of physical parameters to optimize mixing.

Rapid Reconstitution Packages (RRPs) were designed with numerical models integrating microfluidic structures to dramatically enhance mixing of large payloads to optimize mixing in the 1-7 sec range. RRPs were designed using Computational Fluid Dynamics (CFD) models that incorporate API and diluent parameterization of physical properties. CFD modeling provided insights into the flow distribution variables of drug dissolution, such as turbulence, solubility and shear stress.

Device performance was evaluated by selecting Tissue Plasminogen Activator (tPA) as the initial model drug. tPA is a commonly used thrombolytic for treating ischemic stroke and myocardial infarction (MI) [8, 9]. tPA is a large peptide-based molecule, reportedly unstable in liquid form (up to 8 hours in solution) [10]. The drug was selected for investigation as it is normally administered in emergency situations, requiring refrigeration and not able to be reliably stored in liquid form. The tPA used for experiments was Cathflo Activase recombinant (Genentech, Inc.). The present invention may be used with drugs or powders (foods, cosmetics, mixers for pediatric drugs).

RRPs disclosed herein were designed from a clinical perspective to improve logistics for emergency and ambulatory applications in a single-step process. RRPs can potentially provide an impact on the delivery of APIs in wide variety of applications for emergency and ambulatory settings.

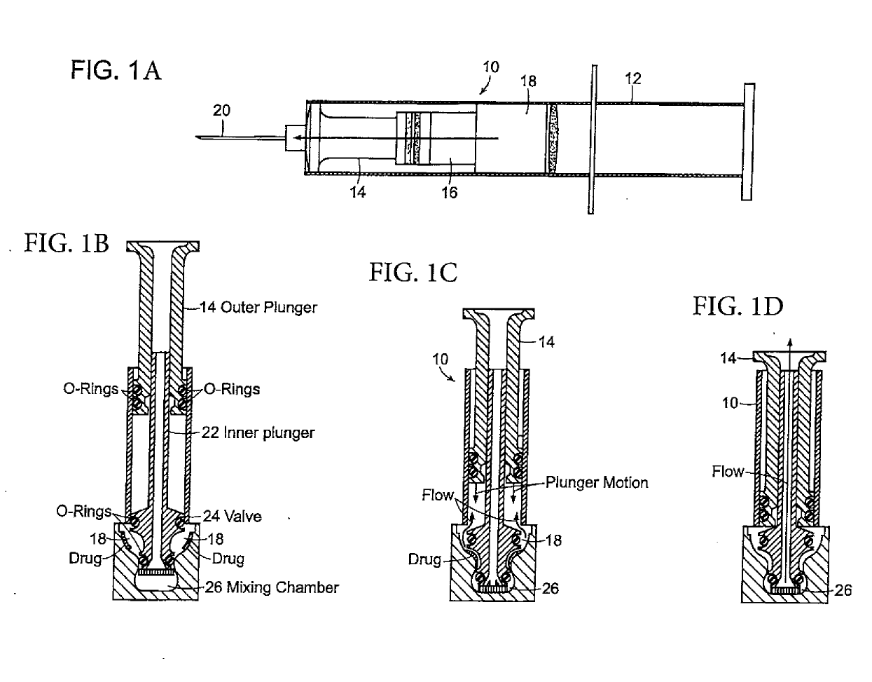

FIG. 1a is an isometric view of an embodiment 10 of the rapid reconstitution package disclosed herein. In FIG. 1a, the rapid reconstitution package 10 is shown disposed within a standard syringe 12. When the syringe 12 plunger is depressed, an outer plunger 14 telescopes or retracts within the RRP 10 causing diluent in a diluent chamber 16 to flow into a drug chamber 18 and then out through the needle portion 20 of the syringe 12. Note that in this embodiment the direction of motion of the RRP plunger 14 is opposite to the direction of motion of the syringe 12 plunger (telescopic back-loop). This arrangement improves mixing and prevents bubble formation as long as the exit tube is prefilled with some diluent and the drug chamber 18 is compartmentalized between two sets of o-rings.

As shown in FIG. 1b, as the RRP telescopes, an inner plunger 22 urges into an open position a valve 24 allowing diluent to flow into the drug chamber 18. If desired, a mixing chamber 26 may be provided to enhance mixing. As will be discussed below, computational fluid dynamics techniques are used to tune the shape of the drug chamber 18 to enhance mixing behavior.

The RRP design disclosed herein was initially conceived as the cylindrical cartridge 10 that can be activated by inserting into a standard syringe 12 and compressed with the syringe plunger. This mode of operation was selected to help preserve the standard operating procedure of drug delivery using syringes. RRPs are designed to fit in 10, 20 and 50 mL syringes, depending on the target pharmacological therapy that corresponds to payload volume. The RRP was designed for activation in three steps. First, the syringe plunger is removed from the syringe barrel. Second, the RRP is placed inside of the syringe barrel as a cartridge. Finally, the syringe plunger is reinserted into the syringe barrel and put into firm contact with the RRP. The system is then ready for operation. The device relies on the telescopic operation of an internal plunger which opens a valve between the diluent and drug chambers, forcing the diluent into the drug chamber. The drug-diluent product is then further mixed in the mixing chamber and then released.

In terms of materials, the device disclosed herein can be made out of polymers or glass (when possible), or a combination of both, for example by deposition of a conformal layer of SiO2 along the surface area of polymers. In addition, Paralyne could be used as an example of a conformal film deposited to prevent leeching or material interference with the drug (API) or diluent.

In terms of manufacturing, as a method, it could be implemented with 3D printing or Stereo Lithography Apparatus (SLA), but ultimately injection molding or other mass production will be needed. The inner back-loop exhaust cylindrical channel (tube) can be defined quite small such that in the case the RRP is implemented as a syringe cartridge, when the end tip of the inner plunger can be inserted without effort as close as possible inside of the syringe inner channel that accesses the needle, preventing any overhead volume and reduces bubbles.

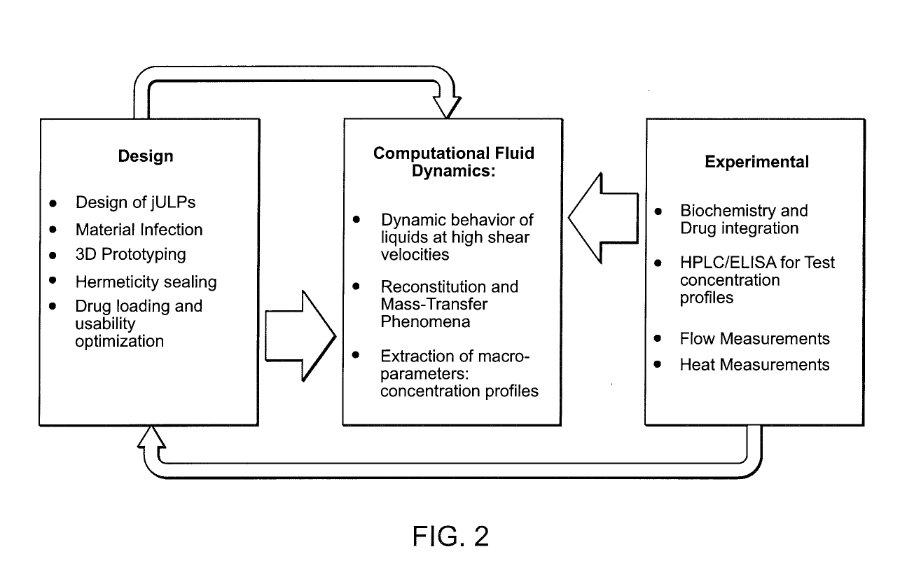

Computational Fluid Dynamics (CFD) techniques were employed to quantitatively optimize RRP reconstitution performance. FIG. 2 shows a diagram illustrating how design, CFD, and experiments were all influenced and informed by each process. The use of CFD provides a set of powerful quantitative techniques for gaining insights into the design of structures intended to optimize mixing parameters for drug mixing, advection, and diffusion phenomena [11-16]. The numerical simulations were performed on the fluid modeling platform Flow-3D (Flow Science, Inc.), which uses a Finite Volume Method combined with a Volume of Fluid Method for free surface tracking [17, 18]. For modeling turbulence, a κ-ε model with Re-Normalization Group (RNG) was adopted. This approach allowed the observation of different dimensional scales present in the simulation, providing predictions for mass transfer [19, 20]. Additionally, a second order projection method was used for solving Navier-Stokes equations in transient analysis with a second order scalar transport model.

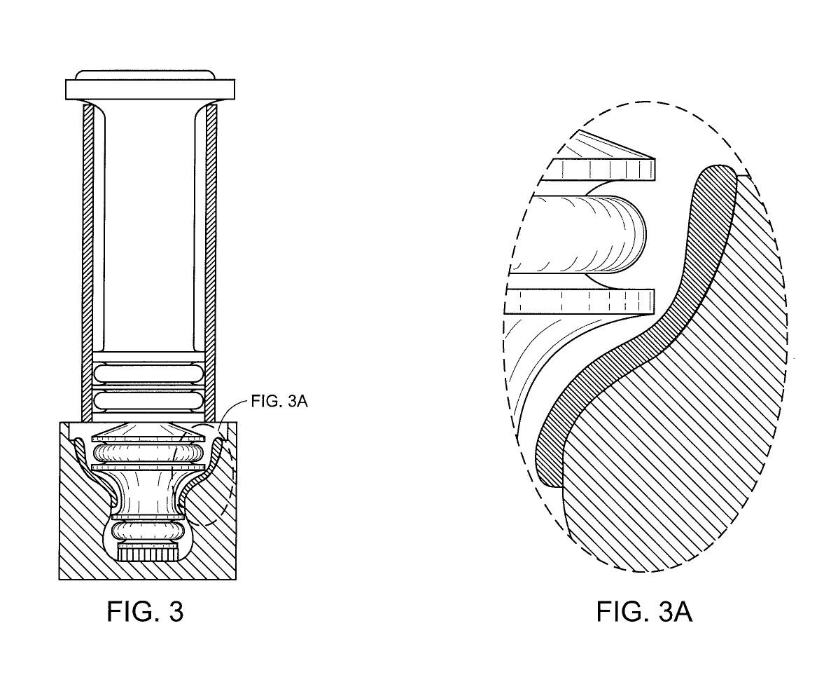

Considering device symmetry, simulations were run on a 1/12^{th} cylindrical domain, at the expense of neglecting angular flow to limit computational load since the speed of simulations for rapid iteration cycles was desirable. The mesh adopted for simulations is shown in FIG. 3, which included approximately 10⁶ finite volumes. Boundary conditions were defined as fixed velocity at input, no slip at the walls and fixed pressure at the output. The resulting average time step for optimized convergence was approximately 30 ms.

Preliminary analyses focused only on diluent flow across structures. The primary concerns for these simulations were increasing inlet velocity into the drug chamber, minimizing stagnation-prone regions, and streamlining the fluid flow for increasing advection. Inlet velocity information was utilized to increase turbulence and vorticity in the drug chamber for improving mixing. Stagnation-prone regions were to be avoided to maximize drug exhaustion. Streamlining was crucial for the overall design optimization.

The drug chamber shape is tuned per physical properties of the drugs, excipients and diluents, including: solubility, packing factor, viscosity of the diluent, viscosity of the product. The size of the drug chamber depends on payload; the size of the diluent chamber depends on required volume to dilute. Furthermore, in case that simulations indicate that a higher viscosity (effective one) is required, one can add nano-particle or micro-particles either or both in the diluent or drug chamber as excipients that enhance reconstitution by means of convective forces. Suitable nano- or micro-particles are PLGA, silica, iron oxide and other biocompatible materials.

Furthermore, one can potentially encapsulate the API with nano-particles or micro-particles that will enhance the reconstitution process by creating nano or micro domains of hydrophilicity. Hydrophobicity of drugs is one of the key physical properties to consider. The challenge in the simulations is to combine convection and diffusion at once. As the drugs are being ‘hit’ by the diluent (with or without jetting) at high velocity, the drugs start to be dissolved as they are mechanically moved down to the drug chamber (convection). This invention could be used as cartridge, injector, or auto-injector.

A more comprehensive CFD model with diluent-drug interactions was introduced to simulate the drug reconstitution process in the preliminarily simulated designs. Physical parameters of the drug and excipients were incorporated into the model.

Cathflo Activase in its delivered form was composed of a 1:40 API to excipient, with 2 mg tPA and 78 mg excipients (sucrose, L-arginine, polysorbate 80, and phosphoric acid), summing up to 80 mg per payload. The entire payload was modeled as a homogeneous compound of sucrose (the major component) with a diffusivity value in water equal to 5.10⁻⁸ m²/s and a mass density of 1.587 mg/mm³. A homogenous mixture of tPA and excipient was assumed to quantify API output concentration as 1/40^{th} of the total output scalar concentration.

The packing factor due to lyophilization was taken into account to incorporate the stacking of the drug into a singular annular volume defined in the drug chamber. The packing factor was estimated and approximated as follows [21]:

$$PF = \frac{{NV}_{sphere}}{{V}_{drug}} = π / \sqrt{18} \approx 0.74 \qquad ( 1 )$$

Where N is the number of crystals, Vₛₚₕₑᵣₑ is the crystal volume approximated as sphere of diameter, d, and V_{drug} is the volume of the drug, distributed as a ring in the drug chamber. The density of the lyophilized drug taking into account the packing factor was estimated at 1.2 mg/mm³.

Distilled ultrapure water was selected as the model diluent. The modeled water was assumed to be Newtonian and incompressible. Gravity and surface tension effects were neglected for the Newtonian fluid as the Froude number, defined as the ratio of inertia to gravity forces, and Weber number, defined as ratio of inertia to surface tension forces, were both much larger than unity.

The effect of flow on drug dissolution upon initial device activation was introduced as a change in effective viscosity, estimated by approximating the resulting solution as a dilute suspension of small rigid spherical particles, estimated as follows [22]:

$${μ}_{eff} = {μ}_{f} \frac{1}{{( 1 - ϕ )}^{2.5}} ≅ {μ}_{f} ( 1 + 2.5 ϕ ) \qquad ( 2 )$$

Where μ_{eff} is the effective viscosity of the diluent, μ_{f} is the viscosity of the fluid without solute and φ is the volume fraction, defined as the diluent volume to the mixed volume.

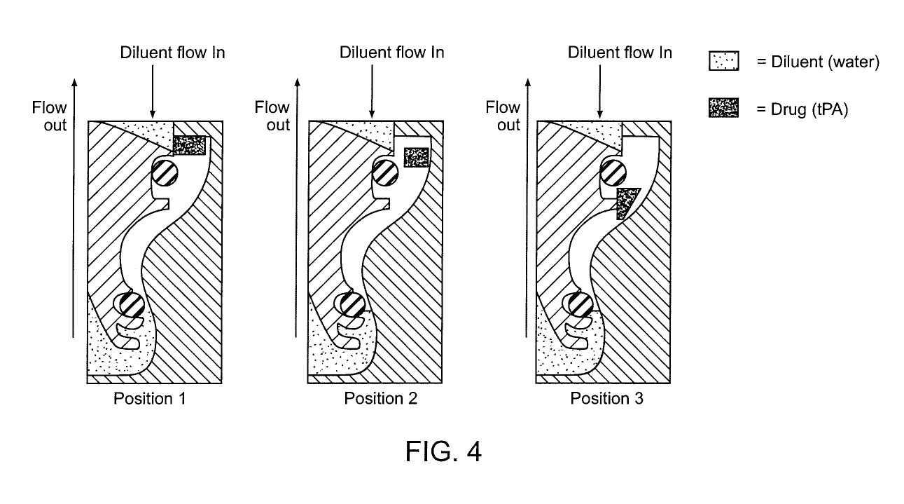

To assess the device behavior under possible operation conditions, numerical simulations were carried out for three different locations of the drug (tPA) within the drug chamber and four distinct activation times of the RRP defined as follows: 1 s, 2 s, 4 s and 7 s. These activation times were chosen to emulate real operation times of syringes. The location of drug was modeled at different positions to account for its random distribution within the drug chamber. The three different positions are shown in FIG. 4 as cross sectional areas of a ring. Position 1 represents the location of the drug RRP on top of the chamber. Position 2 represents the middle position of the drug with the highest probability of being located without any external force acting on it. Position 3 represents the location of the drug RRP on the bottom of the chamber. The activation time was defined from the time of valve opening to the full compression of the internal plunger. A data probe to measure concentration gradients as function of time was defined at the outlet of the RRPs in the simulations.

RRPs were initially kept at 20° C., and subsequently subjected to a fixed wall temperature of 65° C. for 3 hours. Samples were kept in 6 different storage conditions for 24 hours prior to assay preparation: (1) Standard Cathflo Activase lyophilized in glass vial at 20° C.; (2) Cathflo Activase reconstituted in Millipore filtered water and kept in glass vial at 20° C.; (3) lyophilized Cathflo Activase loaded into RRPs and kept at 20° C.; (4) Cathflo Activase lyophilized in glass vial at 65° C.; (5) Cathflo Activase reconstituted in Millipore filtered water and stored in glass vial at 65° C., and (6) Cathflo Activase loaded into RRP and kept at 65° C. After 24 hours each sample in lyophilized form, including those stored in the RRP, was reconstituted in 2 mL Millipore water, and apportioned for subsequent use in ELISA, UV-Vis and HPLC assays.

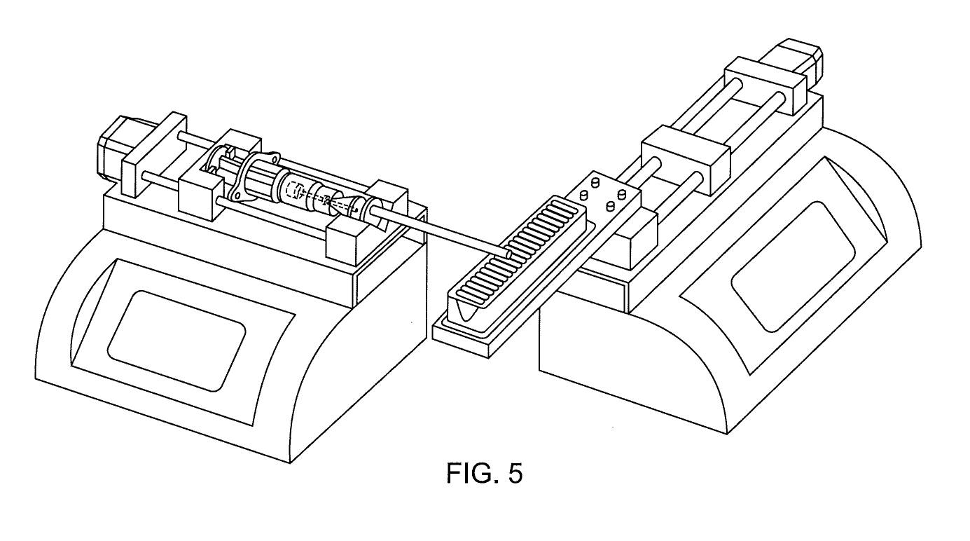

RRPs were loaded with Cathflo Activase (2 mg of tPA and 78 mg of excipients). RRPs were placed in a 25 cc syringe (Exel International, Co.). The syringe was then fixed into a syringe pump (Customized 4X PhD Series, Harvard Apparatus, Inc.) that would activate the RRP at a rate of choice. The syringe pump was set to inject a standard 20 cc volume syringe at a flow rate of 260 mL/min for a total inject time of two seconds. As the flow began to emerge from the outlet of the syringe, a customized linear actuator collected the elution. This linear actuator was configured with a collection tray consisting of 14 wells with a fixed width, such that each well represented a time increment of approximately 0.14 s. An illustration of the experimental setup is shown in FIG. 5. The concentrations of drug in each well were analyzed using a UV spectrometer (Model: 8453 UV-Vis, Spectroscopy System, Agilent Technologies, Inc.) at 210 and 280 nm wavelength with a 100 μL microcuvette (Type 701M10.100B, NGS Precision, Inc.). Concentration for each individual flow increment was measured for multiple trials. The data for all trials was then compiled and compared against a calibration curve, and the average concentration profile of tPA in the eject was derived.

Stability experiments were performed to characterize and compare the RRP with standard vials at various storage conditions. ELISA assays were used to test for stability of samples (Human tPA Platinum ELISA Kit, eBioscience, Inc.) that underwent two temperature conditions, T=20, 65° C. HPLC assays were not sufficient in assaying stability for tPA as the low pH buffer required denatured tPA regardless of conformation or partial denaturation already induced by the storage condition.

The samples to be tested were injected into each ELISA well and left to incubate. After a wash step, a second antibody, horseradish peroxidase (HRP)-conjugated anti-tPA, was injected into each sample well. The HRP-conjugated anti-tPA bound onto the opposing side of the tPA molecule already bound onto the plate-fixed antibody. After a second wash step, a color inducing substrate (TMB substrate) was then injected into the wells, which bound onto the HRP to induce a measureable gradient representing the amount of tPA bound. In order to prevent oversaturation of the antibodies, the samples were diluted to 10 ng/mL to fall within the assay sensitivity range of 2 pg/mL-10 ng/mL. Optical signal intensity from assay plates were measured with spectrophotometer at wavelength 450 nm (PowerWave HT Microplate Spectrophotomer, BioTek Instruments, Inc.). Each sample was normalized against a calibration curve derived from a 5-parameter logistic fit of tPA standards ran concurrently with the samples.

HPLC (1100 Model, Agilent Technologies, Inc.) was performed with each sample referenced against a calibration curve for the drug being measured. Assays were used with a reversed-phase column (ZORBAX SB-CN, 4.6 mm×250 mm column with 5 μL pores, Agilent Technologies, Inc.). The mobile phase was a gradient with an initial hold for 5 min in a ratio of 70:30:0.1 water:acetonitrile (ACN):trifluoroacetic acid (TFA), brought to 50:50:0.1 water:ACN:TFA over 80 min, and subsequently to 100:0.1 ACN:TFA over 15 min. An acidic environment (created by the use of TFA, pKa=0.23) was necessary to separate tPA from solution. Flow rate was 1 mL/min and injection volume was 250 μL. tPA presence was detected by UV spectrometry at 280 nm.

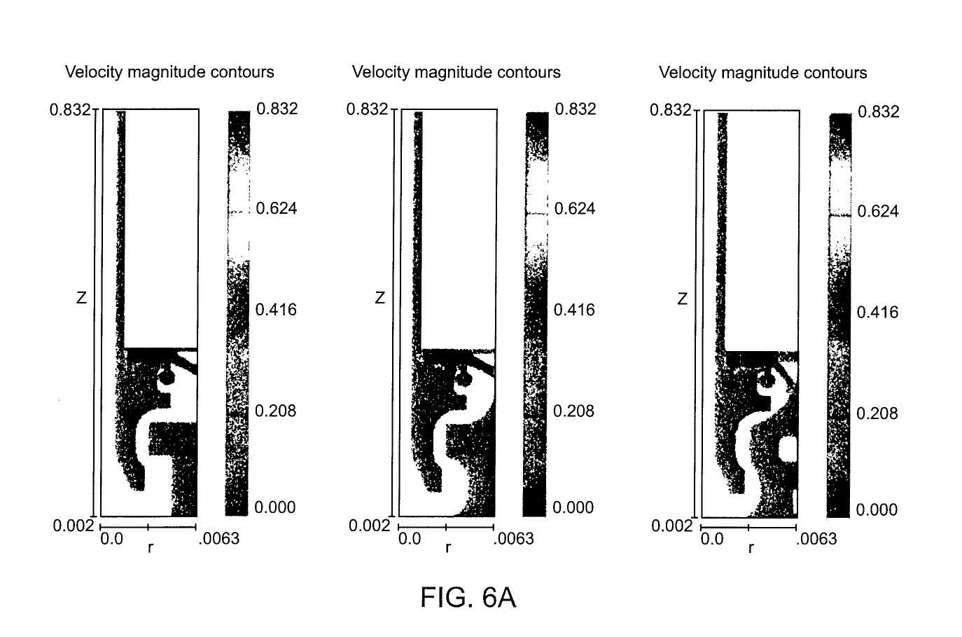

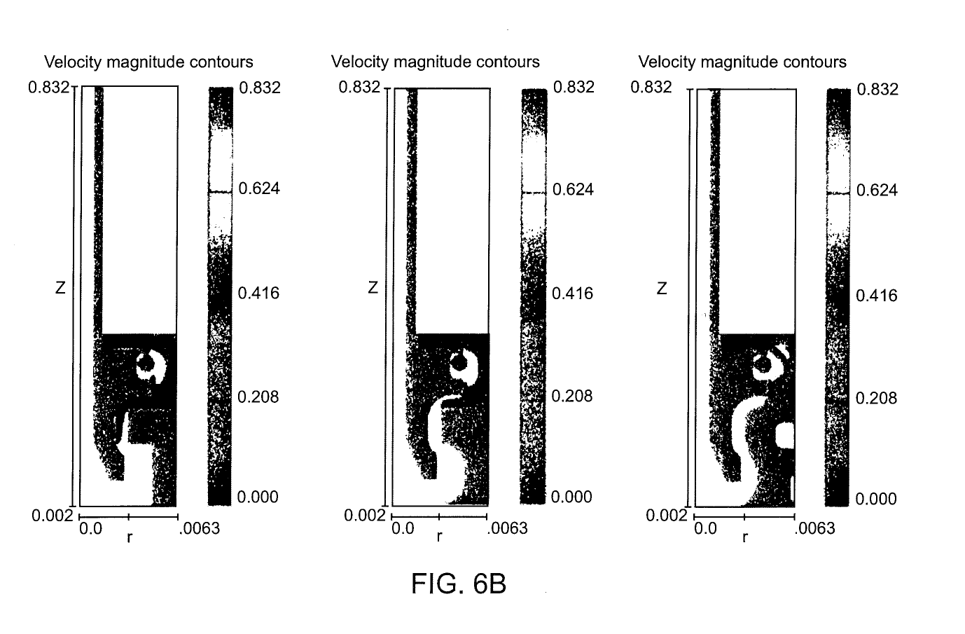

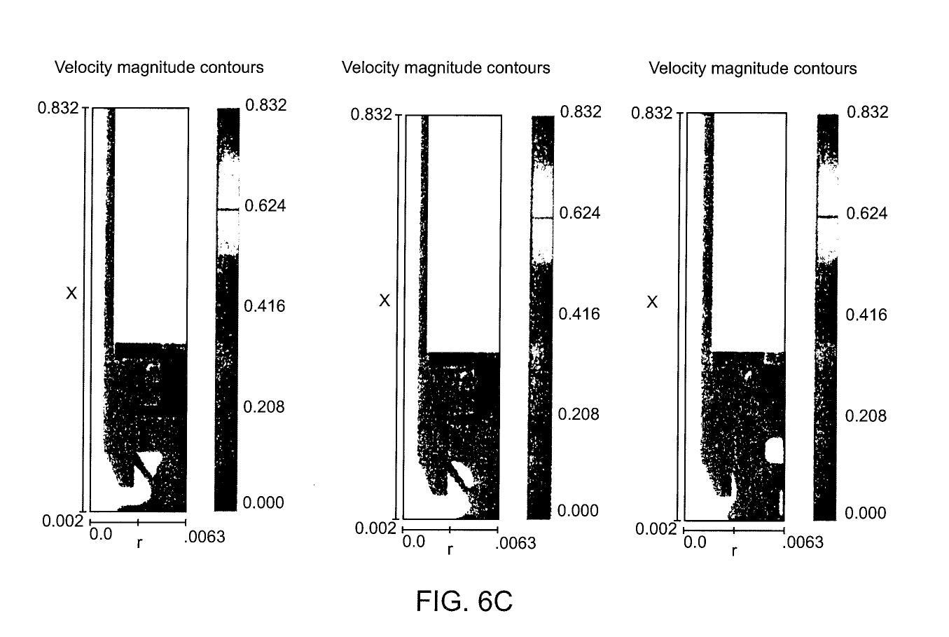

Simulation results with flow characterization models allowed for incremental, yet significant design changes to improve flow properties for three subsequent design versions. FIG. 6a shows the jet effect as a function of changes in the drug chamber structure. Initial design optimization focused on increasing jet velocity, whereas later designs focused on deflecting the direction of the jet downwards for greater mixing in the drug chamber. The change in jet direction corresponded with changes in tuning the drug chamber shape for the three versions, shown in FIG. 6b. These changes were aimed at further increasing reconstitution while eliminating stagnation regions.

Another important aspect is to reduce overhead volume, meaning volume of the diluent, or mixture hung up the in the chambers. There will always be a bit, but also that's another parameter to take into account when optimizing the design. For example, in another proposed embodiment, we can implement a valve that uses sidewall bypasses, maintaining the overall cross section of the device. The way the valve 24 works is that it goes from one small cross section to a larger cross section allowing the o-ring to lose grip from the walls as it enters the drug chamber 18. That is how the jetting effect is included. Because we are adjusting a silhouette and curvature for jetting and mixing, the inner RRP plunger 22 cannot go all the way, and some volume remains. To reduce this, we have changed the valve mechanism. So, instead of going from small cross section to large cross section (barrel), bypasses are spatially defined between barrel in the wall and/or moving plunger. Then, the drug chamber is defined simply straight. A straight (no silhouette) chamber is widely used, e.g. Vetter, etc, but limited to a number of drugs due to solubility and payload. The overhead, volume, however, is much reduced since the inner plunger advances all the way. So, in other words, we can implement a similar concept with telescopic back-loop or without back-loop.

Another significant optimization in the RRP was the elimination of the secondary chamber, which was initially conceived as an additional mixing chamber. This chamber mixing effect was proven insignificant through the simulations. In fact, a large secondary chamber increased the volume of stagnant regions and decreased flow efficiency, as shown in FIG. 6c. The chamber did fulfill a secondary function of separating the drug chamber hermetically from the bottom. Overall internal volume of the RRP was decreased to 63% of the original design.

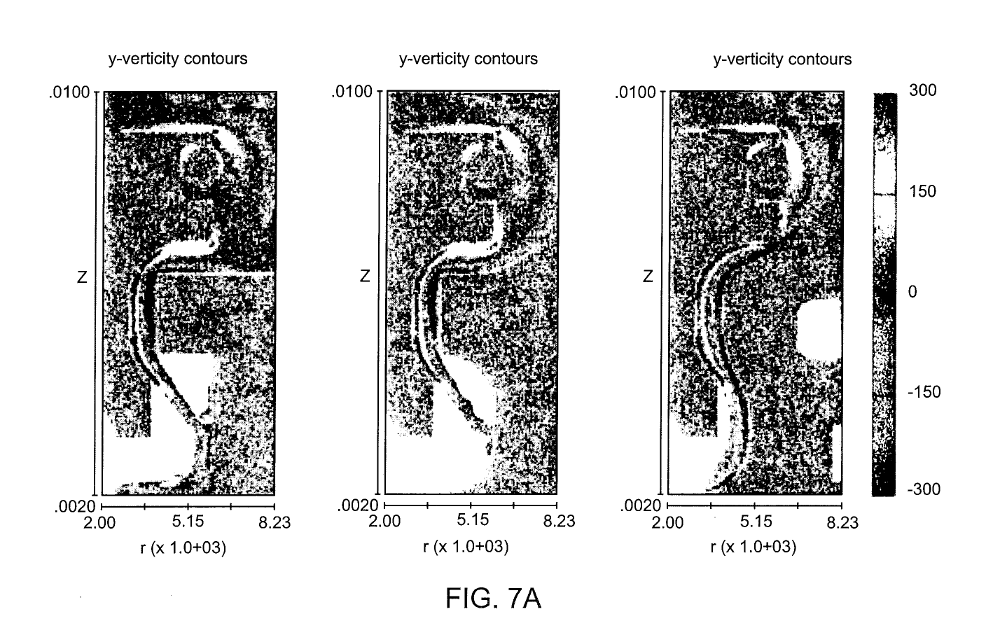

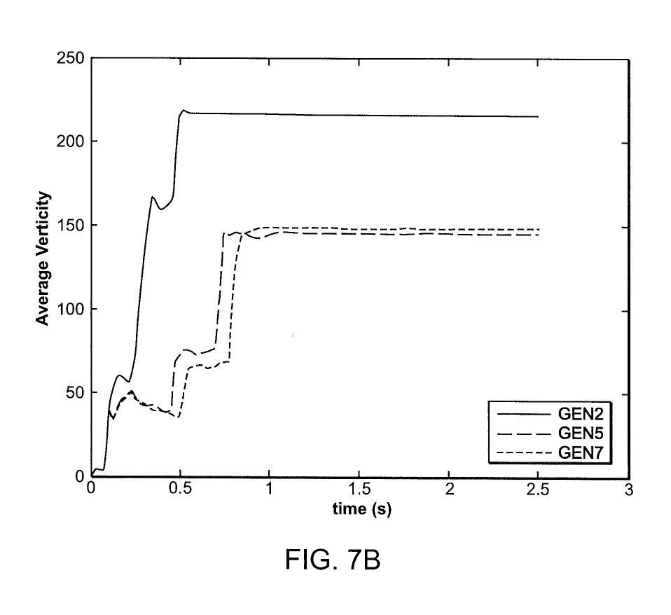

To perform parametric comparison of the designs we define an “average vorticity”, obtained by making a cell volume weighted average of the absolute value of vorticity of the fluid for every fluid cell. A visual representation of average vorticity across the chambers for three subsequent versions is shown in FIG. 7a. This global estimator provided the mixing performance of the device over the operational time, shown in FIG. 7b, demonstrating the impact of design optimization.

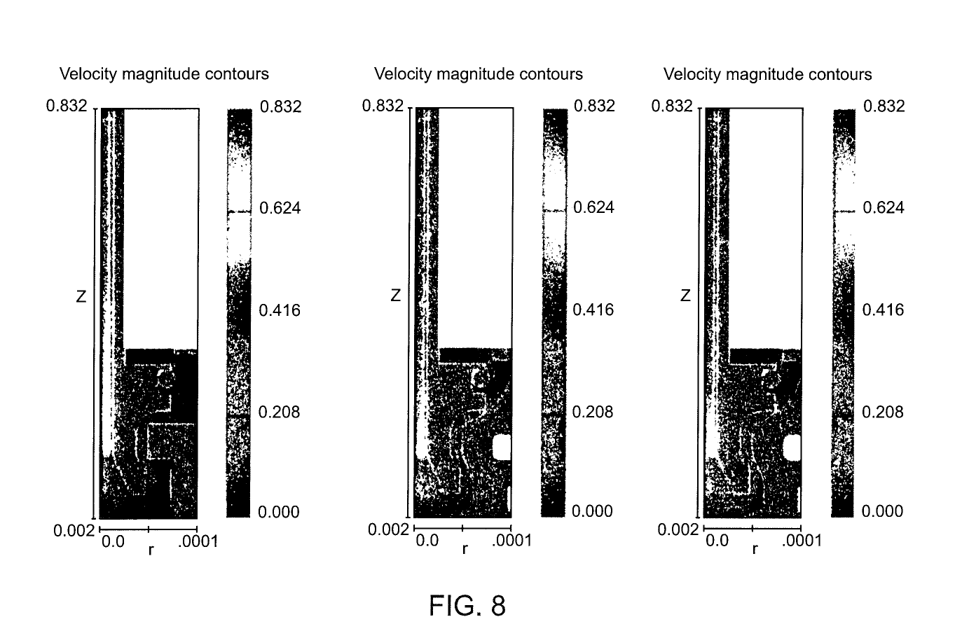

A design requirement that became apparent from observing flow streamlines through the RRP was the addition of a compartmentalized mechanism that would prevent drug powder from exiting the drug chamber too soon. Implementation of such a solution was achieved by adding a secondary valve in order to keep the drug in the drug chamber during the initial transient mixing. FIG. 8 shows the steady state flow pattern where streamlines can be identified.

Simulation results initially showed that a jet of solvent entering the drug chamber collided with the drug crystals as the RRP was activated. This step proved to be a complex process to model due to the combination of drag forces, dissolution and forced convection. Convection was considered the dominant mass transport mechanism for modeling dissolution. The resulting mixed solution was modeled using effective viscosity from Eq. (2) and constant density calculated from Eq. (1). The drug mixing models showed that most of the reconstitution occurred in the drug chamber. The inlet jet effect caused by the opening of the valve between the drug chamber and the diluent reservoir increased reconstitution performance. The fraction of drug that does not exit the chamber dragged by the initial flow goes into localized stagnation regions and slowly diffuses through advection towards the exhaust.

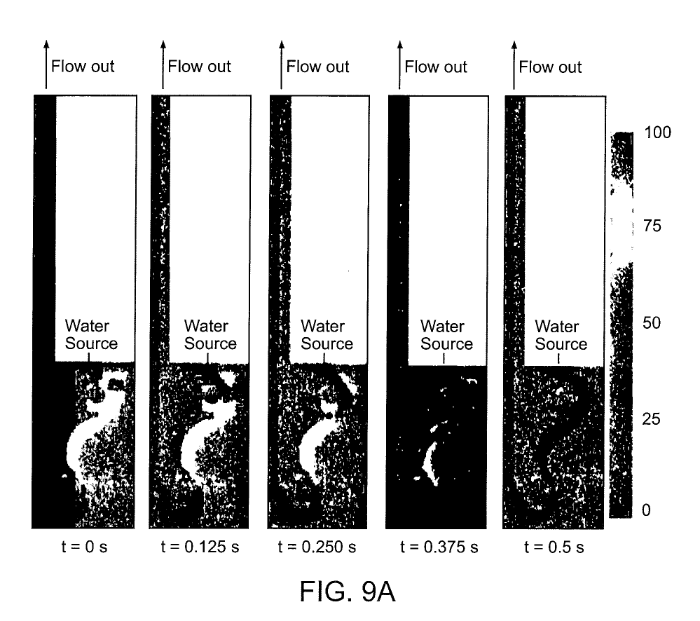

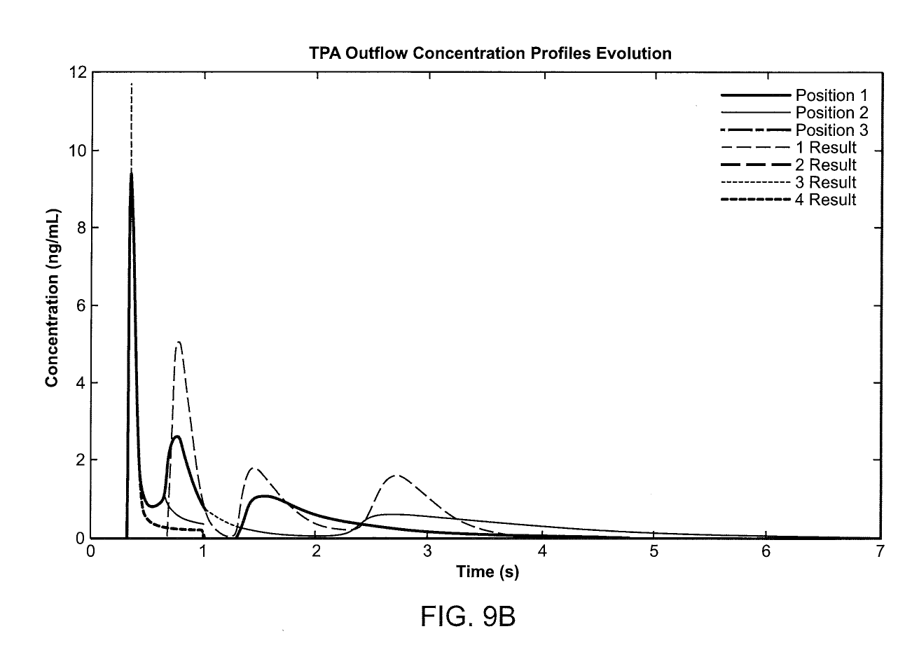

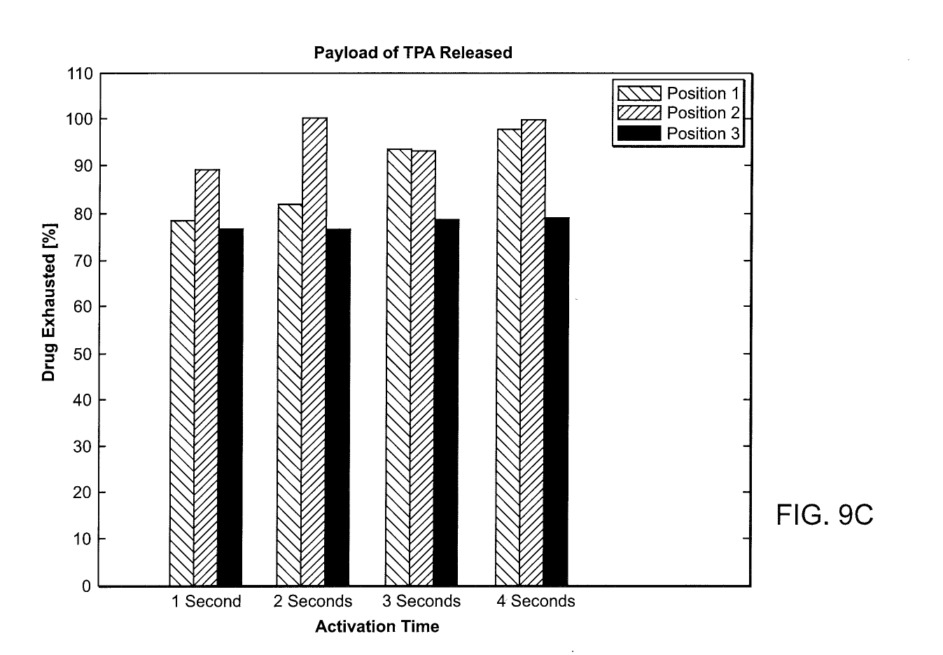

Snapshots of the flow patterns for Position 2 is shown in FIG. 9a as an example of visualization of simulation results. Total results from simulations quantifying concentration eluted from RRPs as a function of time for various locations and injection times are shown in FIG. 9b. The simulated curves resembled Weibull or log-normal distributions, with a larger distribution of drug output towards the onset of activation followed by a tail. These results correlated with subsequent experimental observations, showing that most of the mixing occurred during the initial activation of the RRP with a tail of drug convection. A double peak was observed for the 1 s activation time most likely due to complex flows in the drug chamber caused by the higher velocities. Single peaks were observed for longer activation times (t≧2 s). The output concentration profiles varied significantly with the activation times, indicating that the release kinetics could be significantly tailored by adjusting the input flow rate. The concentration distribution for different drug location runs was more uniform for longer activation times. This effect was attributed to diffusion having a greater impact on drug dissolution as flow rate decreases.

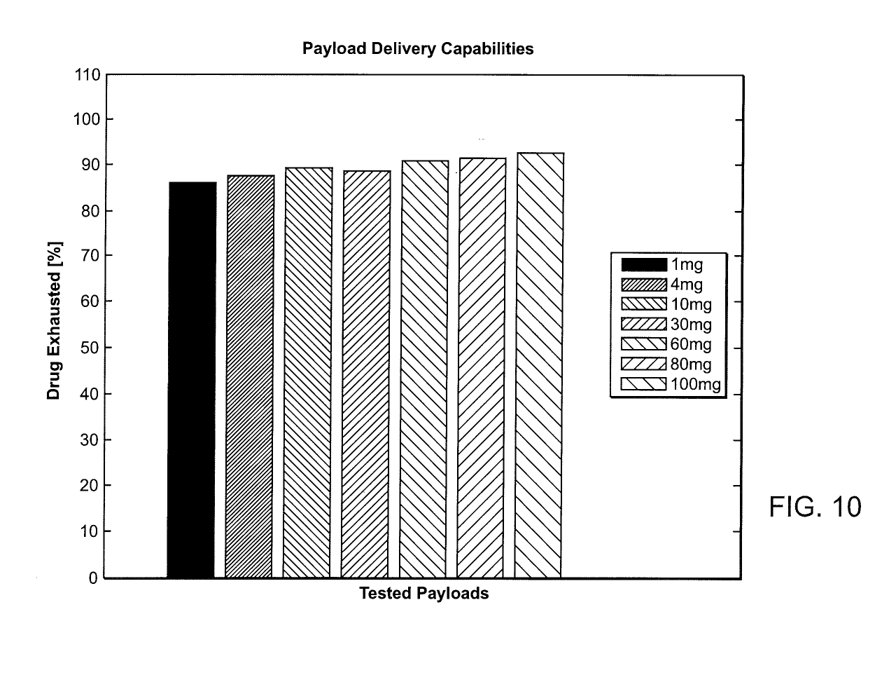

FIG. 9c shows the total amount of drug that was eluted from the RRP after full activation. The total output amount from the RRP was not significantly changed by payload size, initial drug location, or rate of injection, indicating that flow rate may indeed have an effect in concentration profiles but not in the total mass of drug delivered. Simulations for various amounts of tPA were carried out with 1, 4, 10, 30, 60, and 100 mg payloads, which resulted in similar total normalized outputs, as shown in FIG. 10.

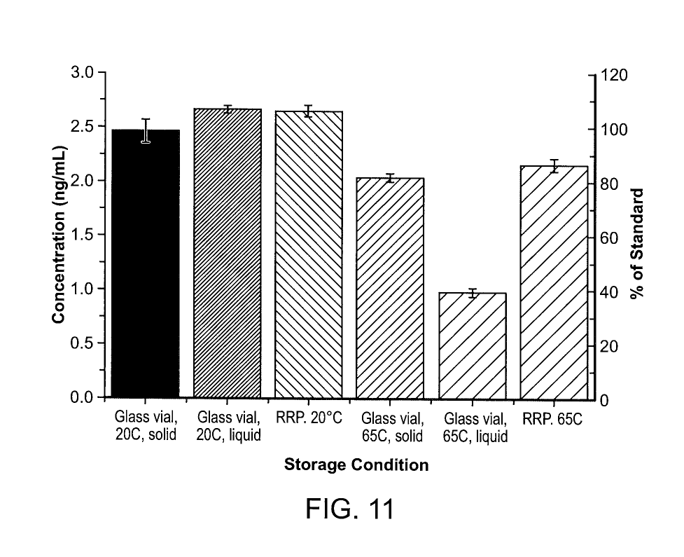

FIG. 11 shows results of ELISA trials of tPA stored in different temperature conditions (T=20, 65° C.). ELISAs showed tPA stability preservation over 24 hours for all samples (solid, liquid, and RRP) at 20° C. tPA stored in reconstituted form within glass vials at 65° C. suffered the greatest loss of activity, expressing only 39.3% in average of the standard activity average. The RRP proved slightly better at retaining drug stability at the critically high temperature of 65° C. than lyophilized drug stored in a standard glass vial with a significant difference, retaining 86.3% in average of total initial drug stability average as opposed to 81.6% average activity in the glass vial.

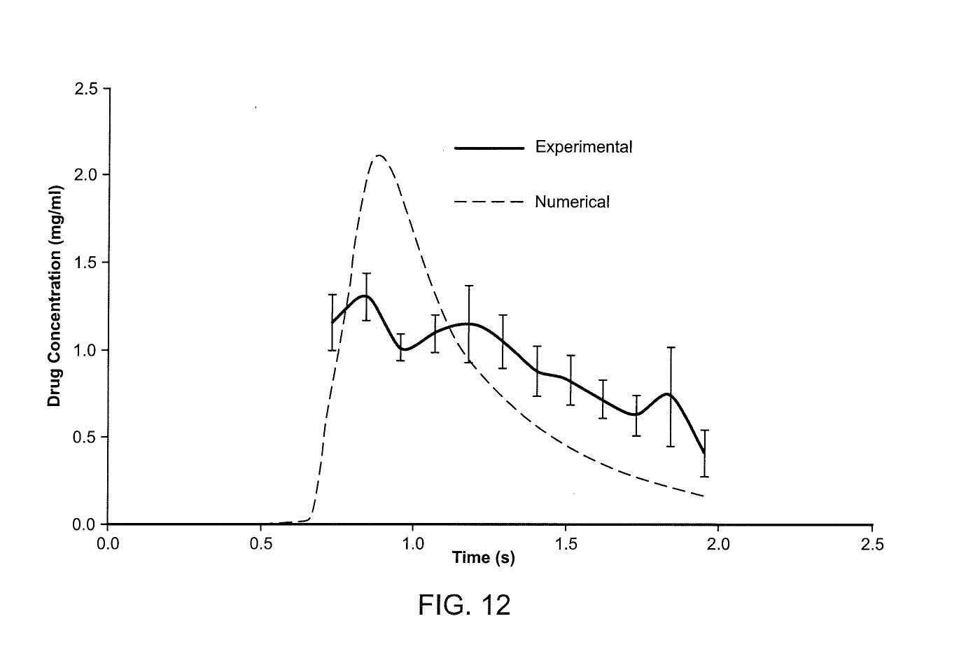

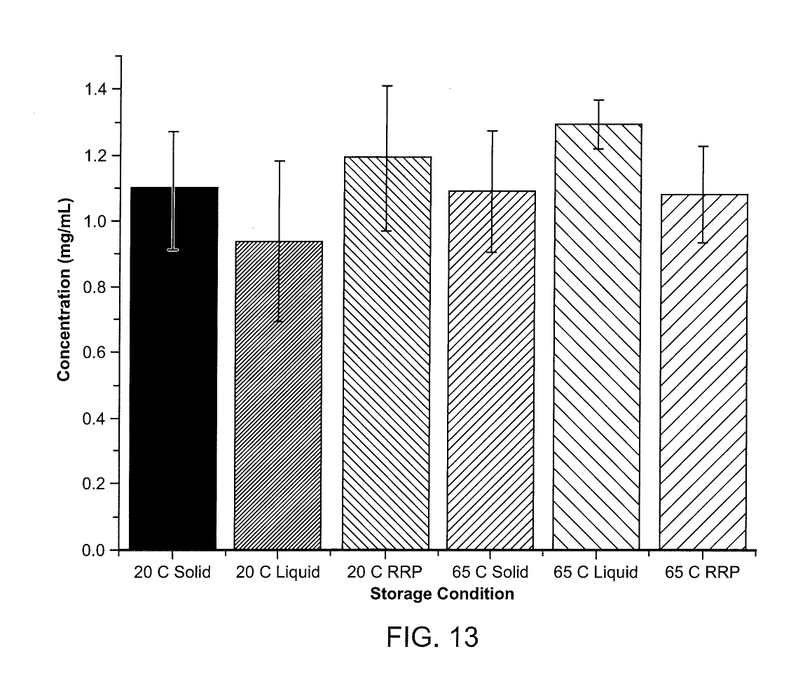

In order to characterize the concentration profile from the RRPs, a 2 s injection time was chosen to emulate a typical injection time as delivered by a clinician. Results from the experimental studies compared to the modeled results are shown in FIG. 12. Experiments revealed that drug became allocated mostly on the sidewalls and bottom of the drug chamber. Consequently, Position 3 was adopted for the simulation as the most representative drug position within the drug chamber, and subsequently incorporated into the numerical model for correlation analysis with the experimental results. The concentration output of all samples analyzed by HPLC did not show a significant difference in variability, as shown in FIG. 13.

The RRP design was optimized by numerical models using CFD models ultimately to maximize reconstitution. The numerical models allow simulations of reconstitution performance for specific pharmacological formulation with different injection times and payloads. Experimental results showed the reliability of simulated results, within the same order of magnitude, in concentration outputs as a function of response time. Differences can be attributed to errors in the discrete sample collection method, primarily related to variability in volume amounts per sample. Another source of variability can be attributed to the slightly different locations of lyophilized APIs between different generations of RRPs, as the drug powder cannot be localized precisely within the drug chamber. A continuous measurement method will be implemented in future work to characterize release kinetics with higher precision.

Each pharmacological formulation requires a set of customized parameters that take into account the physical properties of the API, excipients, and diluents. Experimentally determined concentration profiles are critical for performance and safety. For example, the value of the concentration peak can serve as a limiting factor in the design and optimization of the RRP so it does not reach a specific concentration of toxicity. Conversely, the models can be parameterized to the desired delivery profile in order to inform the design of the fluidic structures. For example, the relative position of the payload within the drug chamber can be considered a design parameter to adjust according to the targeted release kinetics. Simulation results have shown that the Position 3 under activation time of 1 s can be regarded as a bolus application for a drug, whereas Position 1 and activation time of 4 s can be useful for a more continuous release of the same drug.

The payload released has a critical impact in controlling the total dosage delivered in a given pharmacological application. It must be emphasized that the proposed reconstitution model assumes that drug dissolves instantaneously, hence not taking into account drag effects that are experienced by a small portion of solid particles. A more comprehensive model would be required to describe particles that are reconstituted while they are transported.

The presented model allows for simulations results to be incorporated back into the design process, which in turn are quickly implemented using a 3D printing technology. The integrated use of 3D printing technology for the construction of simulated structures provided an advanced approach for accelerating prototyping of microstructures. The computational-experimental iterations provide a method for validating and optimizing reconstitution performance based on structural dimensions and physical parameters. Overall, simulations provide a predictive analytical method, rendering engineering optimization of the RRPs.

Experimental methods including ELISA, HPLC, and flow characterization provided quantitative results required to inform design decisions and ensure design robustness. The integration of CFD models for design of RRPs could be extended to other lyophilized drugs that are required for emergency applications.

The numbers in square brackets throughout this document refer to the references listed herein. The contents of all of these references are incorporated herein by reference in their entirety.

It is recognized that modifications and variations of the present invention will be apparent to those of ordinary skill in the art and it is intended that all such modifications and variations be included within the scope of the appended claims.

### REFERENCES

1. Parshuram C S, T. T., Seto W, Trope A, Koren G, Laupacis A, Systematic evaluation of errors occurring during the preparation of intravenous medication. CMAJ, 2008. 178(1): p. 42-48
2. McDowell S E, M.-I. S., Ashby D, Ferner R E, Where errors occur in the preparation and administration of intravenous medicines: a systematic review and Bayesian analysis. Qual Saf Health Care, 2010. 19: p. 341-345.
3. J K, A., Medication errors: what they are, how they happen, and how to avoid them. Q J Med, 2009. 102: p. 513-521.
4. Wheeler D W, D. B., Sehmi J S, Burnstein R W, Menon D K, Gupta A K, Variability in the concentrations of intravenous drug infusions prepared in a critical care unit. Intensive Care Medicine, 2008. 34(8): p. 1441-1447.
5. Abeysekera A, B. I., Kluger M T, Short T G, Drug error in anaesthetic practice: a review of 896 reports from the Australian Incident Monitoring Study database. Anaesthesia, 2005. 60(3).
6. Pfizer, I., Genotropin: somatropin [rDNA origin] for injection (package insert), Revised August 2006: New York, N. Y.
7. Grassby P F, H. L., Factors affecting the physical and chemical stability of morphine solutions stored in syringes. International Journal of Pharmacy Practice, 1993. 2(1): p. 39-43.
8. Hacke W, K. M., Fieschi C, von Kummer R, Davalos A, Meier D, Larrue V, Bluhmki E, Davis S, Donnan G, Schneider D, Diez-Tejedor E, Trouillas P, Randomised double-blind placebo-controlled trial of thrombolytic therapy with intravenous alteplase in acute ischaemic stroke (ECASS II). Second European-Australasian Acute Stroke Study Investigators. Lancet, 1998. 352(9136): p. 1245-1251.
9. The GUSTO investigators, An international randomized trial comparing four thrombolytic strategies for acute myocardial infarction. New England Journal of Medicine, 1993. 329(10): p. 673-682.
10. Genentech, I., Alteplase (package insert), 2005: South San Francisco, Calif.
11. Garcia E, K. J., Hatch A V, Hawkins K R, Yager P, Controlled Microfluidic Reconstitution of Functional Protein from an Anhydrous Storage Depot. Lab on a Chip, 2004. 4(1): p. 78-82.
12. Bendek A, L. J., Automatic Reconstitution Injector Device, U.S.P.a.T. Office, Editor 2012.
13. May-Newman K, M. M., Lee M N, Design and Preliminary Testing of a Novel Dual-Chambered Syringe. Journal of Medical Devices, 2011. 5(2): p. 037001-1.
14. Kremer D M, H. B., Process simulation in the pharmaceutical industry: a review of some basic physical models. Journal of Pharmaceutical Sciences, 2006. 95(3): p. 517-529.
15. Cortes-Quiroza C A, A. A., Zangeneha M, Gotob A, Analysis and multi-criteria design optimization of geometric characteristics of grooved micromixer. Chemical Engineering Journal, 2010. 160(3): p. 852-864.
16. Broboana D, M. B. C., Wohland T, Balan C, Investigations of the unsteady diffusion process in microchannels. Chemical Engineering Science, 2011. 66: p. 1962-1972.
17. C W, H. Volume-Fraction Techniques: Powerful Tools for Flow Modeling. in Computational Wind Engineering Conference. 1992. University of Tokyo: Flow Science.
18. Isfahani A H G, B. J., On the Implementation of Two-equation Turbulence Models in FLOW-3D®, 2009, Flow Sciences, Inc.
19. Yakhot V, O. S., Renormalization Group Analysis of Turbulence. I. Basic Theory. Journal of Scientific Computing, 1986. 1(1): p. 3-51.
20. Nagano Y, I. Y., Renormalization group theory for turbulence: Assessment of the Yakhot-Orszag-Smith theory. Fluid Dynamics Research, 1997. 20(1-6): p. 152-172.
21. Theodore, L., NanoTechnology: Basic Calculations for Engineers and Scientists 2006, New York: John Wiley & Sons, Inc.
22. Xuan Y, R. W., Conceptions for heat transfer correlation of nanofluids. International Journal of Mass Transfer, 2000. 43: p. 3701-3707.

What is claimed is:

1. Rapid reconstitution package comprising:
a substantially cylindrical structure including a diluent chamber and a drug chamber communicating through a valve, the cylindrical structure configured to retract under axial force causing the valve to open to allow the diluent to flow through the valve into the drug chamber for mixing and reconstitution of the drug, wherein the drug chamber shape is tuned to enhance mixing.

2. The package of claim 1 wherein drug chamber shape is tuned according to physical properties of the drug, excipients and diluents.

3. The package of claim 2 wherein the physical properties include solubility, packing factor, viscosity of the diluent or viscosity of the reconstituted drug.

4. The package of claim 1 further including nano- or micro-particles as excipients in the drug or diluent chambers to enhance reconstitution.

5. The package of claim 1 wherein the drug is encapsulated with nano- or micro-particles to create domains of hydrophilicity.

6. The package of claim 1 wherein the rapid reconstitution package is used as a cartridge, injector or auto-injector.

7. The rapid reconstitution package of claim 1 further including a plunger operatively connected to the valve to open the valve under the axial force.

8. The package of claim 1 wherein the direction of motion of the plunger is opposite to the direction of motion of a syringe plunger.

9. The rapid reconstitution package of claim 1 wherein the cylindrical structure is sized to fit within a standard syringe body.

10. The rapid reconstitution package of claim 1 further including a mixing chamber communicating with the drug chamber to provide enhanced mixing.

11. The rapid reconstitution package of claim 1 wherein the drug chamber shape deflects a jet of diluent downwards for enhanced mixing.

12. The package of claim 11 wherein the jet is tuned in intensity and spatial direction depending on the drug to be reconstituted.

13. Method for designing a rapid reconstitution package comprising:
prototyping and testing physical properties of a package structure;
using computational fluid dynamics to model and numerically process results of the testing; and
using experimental data to modify fluidic structure to achieve enhanced mixing.

14. The method of claim 6 used to modify the silhouette of a drug chamber while monitoring indices of vorticity and concentration as a function of time.

15. The method of claim 13 wherein the computational fluid dynamics provides quantitative techniques to optimize mixing parameters for drug mixing, advection and diffusion phenomena.

16. The method of claim 13 wherein the modified fluidic structure increases inlet velocity into a drug chamber in the package.

17. The package of claim 1 wherein the structure is made of polymer or glass or both.
